(19) United States
(12) Patent Application Publication
(10) Pub. No.: US 2004/0210084 A1
(43) Pub. Date: Oct. 21, 2004

(54) Process for the production of 2,6-naphthalenedicarboxylic acid

(76) Inventors: Gian Luca Castiglioni, Trescore Balneario (IT); Carlo Fumagalli, Albano Sant'Alessandro (IT); Roberto Pirola, Dalmine (IT)

Correspondence Address: Fisher Christen & Sabol, Suite 1108, 1725 K Street, NW, Washington, DC, 20006, US

(21) Appl. No.: 10/488,691

(22) PCT Filed: Sep. 6, 2002
(86) PCT No.: PCT/EP02/10002

(30) Foreign Application Priority Data
Sep. 7, 2001 (EP) 01830573.0

(51) Int. Cl. C07C 51/255

(52) U.S. Cl. 562/414

(57) ABSTRACT

A process for the preparation of 2,6-naphthalenedicarboxylic acid by liquid phase oxidation in acidic solution of 2,6-dimethylnaphthalene in presence of a cobalt-manganese-bromine-catalyst. An oxygen containing feed gas is introduced into the reaction zone such that the oxygen content in the dry exhaust gas does not exceed 1 percent by volume.

The invention relates to a process for the preparation of 2,6-naphthalenedicarboxylic acid (2,6-NDA) in high overall yields and high purity by oxidizing 2,6-dimethylnaphthalene (2,6-DMN) with an oxygen containing feed gas in the presence of a catalyst.

2,6-Naphthalenedicarboxylic acid is an important commercial product, mainly used as a monomer in the production of polyethylene naphthalate (PEN). PEN is a polyester prepared by reacting ethylene glycol and 2,6-NDA or its dialkyl ester and has many important commercial applications like films for magnetic tapes, advanced photo systems and packaging and tyre cords. Potential consumption in packaging is very large. PEN is a higher grade polymer similar to polyethylene therephthalate (PET), produced from ethylene glycol and terephthalic acid. Compared with PET, PEN has better mechanical and thermal resistance and better gas barrier properties.

Efficient production of high quality PEN requires high purity 2,6-NDA and the absence of by products such as 6-formyl-2-naphthoic acid (6-FNA) or 6-methyl-2-naphthoic acid (6-MN) or over-oxidized polybasic acids like trimellitic acid (TMA). TMA, 6-FNA and 6-MN contents have a strong and adverse modifying effect on degree of polymerisation and molecular weight distribution of PEN. Furthermore, TMA forms insoluble complexes with catalyst metal ions and reduces the catalyst content during oxidation step, diminishing the possibility of recycling the reaction mixture after separation of the product from the mother liquors. TMA-metal-complexes precipitated together with 2,6-NDA are difficult to separate from the latter.

To fully exploit the potential market of PEN it is very important to develop a competitive process for the oxidation of the preferred raw material 2,6-DMN to 2,6-NDA in high purity.

EP 439007 A2 discloses a process for the production of 2,6-NDA which comprises oxidizing a 2-alkyl-6-acyl naphthalene with molecular oxygen-containing gas in the presence of a catalyst containing cobalt, manganese and bromine.

EP 439007 A2 starts with a partly oxidized compound and doesn't disclose a procedure for the oxidation of 2,6-DMN. U.S. Pat. No. 3,856,855 discloses a process for the oxidation of mono- and dimethylnaphthalenes which comprises oxidizing the substituted naphthalenes in acetic acid solvent in the presence of a three component catalyst containing defined amounts of a cobalt compound, a manganese compound and a bromine compound.

U.S. Pat. No. 3,856,855 specifies that at temperatures exceeding 180° C. dark colored products are obtained and it is impossible to obtain the intended naphthalenecarboxylic acids in high yields. Low reaction temperatures on the other hand mean also low reaction rates and larger amounts of intermediate oxidation products like carboxy-naphthaldehyde (=6-FNA) and methylnaphthoic acid which are particularly detrimental in the polymerization reactions for which 2,6-NDA is used. Best yields reported for oxidation of 2,6-DMN to 2,6-NDA are at about 86%.

U.S. Pat. No. 4,933,491 is directed to an oxidation process for the oxidation of 2,6-DMN to crude 2,6-NDA (examples 1 and 2) and further purification method for 2,6-NDA (examples 3 to 7). The examples 1 and 2 (each three repetitions) don't mention the oxygen content in the exhaust gas. Results showed in Tables 1 and 2 don't mention possibly different results of the repetitions. TMA content in examples 1 and 2 is 24200 ppm and 8900 ppm respectively.

U.S. Pat. No. 5,183,933 specifies oxidation conditions for the production of 2,6-NDA starting from 2,6-DMN. In all examples oxygen containing gas is supplied so that the oxygen concentration in the exhaust gas is 4 to 6% by volume. All examples feature at least formation of 2.5% (25000 ppm) TMA by-product which has to be removed after the separation of the product. U.S. Pat. No. 5 183 933 states that the presence of TMA among the by-products is very detrimental to the efficiency of the catalyst system, because of the formation of manganese-TMA (Mn-TMA) salts which are insoluble in the reaction medium and increase the consumption of catalyst. Mn-TMA salts form a precipitate with 2,6-NDA and are hard to remove from the product, furthermore this precipitation diminishes the amount of available catalyst and its recovery rate.

U.S. Pat. No. 5,763,648 is directed to a process for the production of terephthalic acid which comprises oxidizing p-xylene with a molecular oxygen-containing gas in the presence of sodium hydroxide and a catalyst containing cobalt, manganese and bromine. In all examples oxygen containing gas is supplied so that the oxygen concentration in the exhaust gas is 6% by volume. Although the oxidation of 2,6-DMN to 2,6-MDA is mentioned as an applicable field of use of the invention the disclosed process provides no solution to problem of over-oxidation and its detrimental effects in the production of PEN.

WO-A1 -98/42649 describes the oxidation of 2,6-DMN in the presence of a catalyst including cobalt, manganese and bromine, with a weight ratio of cobalt to manganese greater than 1. All examples are performed with an oxygen content in dry exhaust gas of 2.5 to 3.5 vol. %. Reduced formation of TMA and less content of metals in dried 2,6-NDA is claimed compared with prior art. However, the amount of TMA in the crude 2,6-NDA is in the range of 2200 to 4500 ppm and the total metal amount is in the range of 1400 to 3200 ppm. Moreover the catalyst is expensive due to the used high cobalt/manganese ratio while cobalt being the most expensive component of the catalytic system.

Several Japanese patent applications have been published concerning the oxidation of 2,6-DMN to 2,6-NDA. JP-A-10-291958 claims a suitable oxygen range of 0.5 to 5 vol. % but the oxygen content measured in all examples is in the range of 1.8 to 2.2 vol. %. JP-A-2000-143583 discloses a batch process, wherein the oxygen content in the dry exhaust gas stream is about 2 vol. % during oxidation and about 10 vol. % at the end of the oxidation process.

GB 1 384 110 describes the oxidation of very diluted solutions of 2,6-DMN in order to obtain good yields and high crude purity. The molar ratio of 2,6-DMN to acetic acid solvent is maintained at least at 1:100 and preferably at least at 1:200. Due to the high dilution the process is disadvantageous from the economic point of view and in spite of the high dilution the yield of TMA is always above 3%.

In all cited patents and patent applications as well as standard publications like W. Partenheimer [Catalysis Today 23(1995) 69-158] an oxygen content in the exhaust gas between 4 to 5% is prefer. None of the cited documents discloses a process for the efficient oxidation of 2,6-MDN to 2,6-NDA with almost complete suppression of the formation of over-oxidized by-products.

The technical problem to be solved by the present invention was to provide a selective and high-yield process for the production of 2,6-naphthalenedicarboxylic acid from 2,6-dimethylnaphthalene avoiding over-oxidation and formation of larger amounts of objectionable by-products, in particular trimellitic acid.

According to the present invention, this problem has been solved by the process of claim 1.

The present invention is directed to a process for the preparation of 2,6-naphthalene-dicarboxylic acid by liquid phase oxidation of 2,6-dimethylnaphthalene, comprising

a) an oxidation step in a first reaction zone comprising reacting a mixture comprising

aa) 2,6-dimethylnaphthalene

ab) a solvent comprising at least

i) an monocarboxylic acid selected from the group consisting of formic, acetic, propionic, butyric or isobutyric acid, benzoic acid and mixtures thereof, and

ii) water

ac) a catalyst system comprising compounds of cobalt, manganese and bromine, and an oxygen containing feed gas,

b) optionally a post-oxidation step in a second reaction zone, and

c) an isolation step of the product 2,6-naphthalenedicarboxylic acid,

wherein during the oxidation step the flow rate of the oxygen containing feed gas introduced into the first reaction zone is regulated in such a way that the oxygen content of the dry exhaust gas does not exceed 1 percent by volume.

The aforesaid first and second reaction zones may be the same or different.

Surprisingly, an oxygen concentration in the exhaust gas which does not exceed 1 vol. % leads to unexpected high-yield formation of high purity 2,6-NDA accompanied by a minimum content of over-oxidized by-products.

The ratio of 2,6-dimethylnaphthalene to solvent in the process is preferably in the range of 1:4 to 1:12 by weight.

Preferably the monocarboxylic acid in the solvent is acetic acid.

In the process according to this invention the reaction mixture may contain water in the range of about 2 to 20% by weight, preferably about 2 to 10% by weight. This includes the amount of water which is formed in the oxidation reaction either in a batch, semicontinuous or continuous process.

Cobalt and manganese compounds can be independently, hydroxides, the salts of mono-carboxylic acids as defined above, inorganic acids, and mixtures thereof.

Preferably salts of inorganic acids of cobalt and manganese may be e.g. halides, nitrates or hydroxides, which are soluble in the solvent.

In a preferred embodiment salts of cobalt and manganese compounds are acetates, bromides or nitrates.

Bromine compounds can be organic bromine compounds, e.g. linear or branched aliphatic bromides containing 1 to 6 carbon atoms, hydrogen bromide, inorganic bromides, or mixtures thereof.

In a preferred embodiment bromine compounds are selected from hydrogen bromide, ammonium bromide, cobalt bromide, manganese bromide and mixtures thereof.

The atomic ratio of cobalt to manganese added to the reaction zones is preferably in the range of 1:2 to 1:5.

The ratio of cobalt to 2,6-dimethylnaphthalene added to the reaction zones is preferably in the range of 0.5 to 2.5% by weight, calculated as elemental cobalt.

The weight ratio of bromine to the sum of cobalt and manganese content added to the reaction zones is preferably in the range of 0.4:1 to 1: 1, calculated as elemental cobalt, manganese and bromine.

The oxygen content in the dry exhaust gas is preferably regulated such that it does not exceed 0.7 vol. %.

The oxygen containing feed gas can be pure oxygen, air, oxygen enriched air, oxygen containing nitrogen or a gaseous mixture of oxygen containing gases.

The total pressure in the reactor may be sufficient to keep the solvent in the liquid phase, preferably in the range of 6 to 28 bar.

For maintaining a suitable reaction rate, directing the selectivity of oxidation to the desired product, avoiding darkening of the reaction product and reducing the combustion rate of the solvent to carbon oxides, reaction temperatures are preferably in the range of 150 to 225° C., more preferably in the range of 190 to 215° C.

The reaction may be carried out in a batch, semicontinuous or in a continuous mode. In the continuous mode after separation of 2,6-NDA, the mother liquors are preferably recycled to the reactor.

The content of 2,6-NDA in the crude product is higher than in known processes. Operating according to the above preferred conditions, it is possible to obtain 2,6-NDA in total yields above 97% with a purity exceeding 99% even in the crude product. Formation of the main by-product TMA is very much reduced to a content less than 200 ppm in the crude 2,6-NDA. Moreover, the metal content in the crude is very low, e.g. at about 100 ppm, and the color of the crude is much lighter than that obtained by processes operating at higher oxygen partial pressures. The low oxygen concentration avoids over-oxidation and reduces formation of TMA. Catalyst activity is preserved and therefore also the amount of partially oxidized by-products like 6-formyl-2-naphthoic acid (6-FNA) and 6-methyl-2-naphthoic acid (6-MN) is reduced.

Precipitated crystals of 2,6-NDA obtained according to the present invention have higher purity and less metal content because of reduced formation of e.g. manganese salts of TMA, which are insoluble in the reaction medium.

All examples disclosed in prior art exhibit an almost 10-fold amount of the by-product TMA and metal residue in the main product 2,6-NDA induced by over-oxidation.

The invention is illustrated by the following non-limiting examples.

All examples in the present invention were carried out in a reactor in which the distance between the inlet for an oxygen containing gas and the surface of the reaction solution in a static state was adjusted to be 7 cm.

### EXAMPLE 1

### 2,6-Naphthalenedicarboxylic Acid

The experiment was performed in a 1 L titanium autoclave equipped with efficient stirring, overhead condenser, return line for the condensate, feeding lines for air and 2,6-DMN, temperature and pressure control, on-line analyzers for oxygen, CO and CO₂ in the exhaust gas.

In the autoclave were introduced 512 g of acetic acid (water content 5% wt), 2.98 g of cobalt acetate tetrahydrate, 9.44 g of manganese acetate tetrahydrate and 1.73 g of ammonium bromide.

The autoclave was closed and nitrogen was fed to remove air. Temperature and pressure were increased to 205° C. and 21 bar under stirring before starting 2,6-DMN and air feed. 57 g of 2,6-DMN, kept in the molten state at 120° C., were fed in two hours by a heated metering pump. Air flow was regulated through a mass flow meter in order to maintain the oxygen concentration in the dry exhaust gas below 0.7 vol. % (average 0.5 vol. %), measured by on-line oxygen analyzer.

After two hours 2,6-DMN feed was stopped and an oxygen containing gas (5 to 8 vol. % oxygen) was fed for 30 minutes. This post oxidation step is well known in the art (e.g.

U.S. Pat. No. 5,183,933) and reduces the amount of partially oxidized compounds like 6-FNA without substantially increasing the amount of TMA in the product.

After cooling down to room temperature and depressurising to ambient pressure, the reaction products were analyzed by High Pressure Liquid Chromatography for organic components. The conversion of 2,6-DMN was complete and molar yields of desired product and by-products were:

| Compound | Yield |
|---|---|
| 2,6-NDA | 97.1% |
| TMA | 1.0% |
| 6-FNA | 0.2% |
| 6-MN | <0.1% |
| others | 1.7% |

After filtration the crude 2,6-NDA filter cake was washed with an equivalent weight of acetic acid containing 5% wt water and dried. The dried solid was analyzed by high pressure liquid chromatography (HPLC) for organic components and by inductively coupled plasma (ICP) to detect the amount of metals. The composition of the dried solid is shown below:

| Compound | Yield | |
|---|---|---|
| 2,6-NDA | 99.3 | wt % |
| TMA | 150 | ppm |
| 6-FNA | 0.11 | wt % |
| others | 0.6 | wt % |
| total metals | 70 | ppm |

### EXAMPLES 2 to 4

### 2,6-Naphthalenedicarboxylic Acid

The general procedure of example 1 was repeated with variations in the composition of the reaction mixture, experimental conditions and results are summarized in Table 1. Mainly the feed gas flow was regulated in a way that the oxygen concentration in the dry exhaust gas was kept below 0.7 vol. %.

### EXAMPLE C1 (Comparative) 2,6-Naphthalenedicarboxylic Acid

The general procedure of example 1 was repeated with variations in the composition of the reaction mixture, experimental conditions and results are summarized in Table 1. Mainly the feed gas flow was regulated in a way that the oxygen concentration in the dry exhaust gas was kept at 4.9 vol. %.

### EXAMPLE C2 (Comparative) 2,6-Naphthalenedicarboxylic acid

The general procedure of example 1 was repeated with variations in the composition of the reaction mixture, experimental conditions and results are summarized in Table 1. Mainly the feed gas flow was regulated in a way that the oxygen concentration in the dry exhaust gas was kept at 6.0 vol. %.

TABLE 1

| Example No. | 1 | 2 | 3 | 4 | C1 | C2 |
|---|---|---|---|---|---|---|
| Reaction parameters | | | | | | |
| AcOH (95% wt) [g] | 512 | 512 | 512 | 512 | 512 | 512 |
| 2,6-DMN [g] | 57 | 43 | 57 | 85 | 57 | 85 |
| AcOH/2,6-DMN [wt/wt] | 9 | 11.9 | 9 | 6 | 9 | 6 |
| Co(OAc)₂.4 H₂O [g] | 2.98 | 3.27 | 2.17 | 2.98 | 2.98 | 2.45 |
| Mn(OAc)₂.4 H₂O [g] | 9.44 | 10.36 | 10.30 | 9.44 | 9.44 | 7.77 |
| NH₄Br [g] | 1.73 | 1.90 | 1.73 | 1.73 | 1.73 | 1.42 |
| Temperature [° C.] | 205 | 215 | 205 | 205 | 205 | 195 |
| Pressure [bar] | 21 | 21 | 21 | 21 | 21 | 21 |
| O₂ in exhaust gas [vol. %] | 0.5 | 0.65 | 0.6 | 0.6 | 4.9 | 6.0 |
| Conversion [%] | 100 | 100 | 100 | 100 | 100 | 100 |
| Yields in reaction mixture | | | | | | |
| 2,6-NDA yield [mol %] | 97.1 | 95.9 | 97.0 | 96.5 | 93.8 | 94.4 |
| Content in dried crude | | | | | | |
| 2,6-NDA [mol %] | 99.3 | 99.0 | 99.3 | 99.1 | 98.6 | 97.6 |
| TMA [ppm] | 150 | 0 | 130 | 114 | 1820 | 15400 |
| 6-FNA [mol %] | 0.11 | 0.11 | 0.11 | 0.17 | 0.11 | 0.25 |
| Others [mol %] | 0.6 | 0.9 | 0.6 | 0.7 | 1.1 | 0.6 |
| Total metals [ppm] | 70 | 90 | 118 | 69 | 650 | 4950 |

1. Process for the preparation of 2,6-naphthalenedicarboxylic acid by liquid phase oxidation of 2,6-dimethylnaphthalene, comprising:
(a) an oxidation step in a first reaction zone comprising reacting a mixture comprising
(aa) 2,6-dimethylnaphthalene
(ab) a solvent comprising
(ii) an monocarboxylic acid selected from the group consisting of formic, acetic, propionic, butyric or isobutyric acid, benzoic acid and mixtures thereof, and
(iii) water
(ac) a catalyst system comprising compounds of cobalt, manganese and bromine, and an oxygen containing feed gas,
(b) optionally a post-oxidation step in a second reaction zone, and
(c) an isolation step of the product 2,6-naphthalenedicarboxylic acid, wherein during the oxidation step the flow rate of the oxygen containing feed gas introduced into the first reaction zone is regulated in such a way that the oxygen content of the dry exhaust has does not exceed 1 percent by volume.

2. The process of claim 1, wherein the ratio of 2,6- dimethylnaphthalene to solvent is in the range of 1:4 to 1:12 by weight.

3. The process of claim 1, wherein the monocarboxylic acid is acetic acid.

4. The process of claim 1, wherein the reaction mixture contains water in the range of 2 to 20 percent by weight, preferably 2 to 10 percent by weight.

5. The process of claim 1, wherein the cobalt and manganese compounds are independently, hydroxides, the salts of an acid selected from the group consisting of formic, acetic, propionic, butyric and isobutyric acid, benzoic acid, inorganic acids and mixtures thereof.

6. The process of claim 5, wherein the salts are selected from the group consisting of hydroxides, acetates, balides, nitrates, preferably acetates, bromides or nitrates.

7. The process of claim 1, wherein the compounds of bromine are selected from the group consisting of linear or branched aliphatic bromides containing 1 to 6 carbon atoms, hydrogen bromide, inorganic bromides and mixtures thereof.

8. The process of claim 1, wherein the atomic ration of cobalt to manganese added to the reaction zone, is in the range of 1:2 to 1:5.

9. The process of claim 1, wherein the ratio of cobalt to 2,6-dimethylnaphthalene added to the reaction zone is in the range of 0.5 to 2.5 percent by weight, calculated as elemental cobalt.

10. The process of claim 1, wherein the weight ratio of bromine to the sum of cobalt and manganese content added to the reaction zone, calculated as elemental manganese, cobalt and bromine is in the range of 1:0.4 to 1:1.

11. The process of claim 1, wherein the flow ratio of the oxygen containing feed gas introduced into the reaction zone is regulated in such a way that the oxygen content in the dry exhaust gas does not exceed 0.7 vol. percent.

12. The process of claim 1, wherein the oxygen containing feed gas is selected from the group consisting of air, oxygen enriched air, oxygen enriched nitrogen and gaseous mixtures of oxygen containing gases.

13. The process of claim 1, wherein the total pressure is 6 to 28 bar.

14. The process of claim 1, wherein the reaction temperature is 150 to 220° C., preferably 190 to 215° C.

15. The process of claim 1, wherein the reaction is carried out in a continuous mode and the mother liquors obtained in the isolation step are recycled to the reactor.

16. The process of claim 2, wherein the monocarboxylic acid is acetic acid.

17. The process of claim 16, wherein the reaction mixture contains water in the range of 2 to 20 percent by weight, preferably 2 to 10 percent by weight.

18. The process of claim 17, wherein the cobalt and manganese compounds are independently, hydroxides, the salts of an acid selected from the group consisting of formic, acetic, propionic, butyric and isobutyric acid, benzoic acid, inorganic acids and mixtures thereof.

19. The process of claim 6, wherein the compounds of bromine are selected from the group consisting of linear or branched aliphatic bromides containing 1 to 6 carbon atoms, hydrogen bromide, inorganic bromides and mixtures thereof.

20. The process of claim 19, wherein the atomic ratio of cobalt to manganese added to the reaction zone, is in the range of 1:2 to 1:5.

21. The process of claim 20, wherein the ratio of cobalt to 2,6-dimethyinaphthalene added to the reaction zone is in the range of 0.5 to 2.5 percent by weight, calculated as elemental cobalt.

22. The process of claim 21, wherein the weight ratio of bromine to the sum of cobalt and manganese content added to the reaction zone, calculated as elemental manganese, cobalt and bromine is in the range of 1:0:4 to 1:1.

23. The process of claim 22, wherein the flow rate of the oxygen containing feed gas introduced into the reaction zone is regulated in such a way that the oxygen content in the dry exhaust gas does not exceed 0.7 vol. percent.

24. The process of claim 23, wherein the oxygen containing feed gas is selected from the group consisting of air, oxygen enriched air, oxygen enriched nitrogen and gaseous mixtures of oxygen containing gases.

25. The process of claim 24, wherein the total pressure is 6 to 28 bar.

26. The process of claim 25, wherein the reaction temperature is 150 to 220° C., preferably 190 to 215° C.

27. The process of claim 26, wherein the reaction is carried out in a continuous mode and the mother liquors obtained in the isolation step are recycled to the reactor.
